(19) United States
(12) Patent Application Publication
(10) Pub. No.: US 2024/0110158 A1
(43) Pub. Date: Apr. 4, 2024

(54) METHOD FOR SEPARATING AND EXTRACTING SUBPOPULATIONS OF GASTRIC CANCER TISSUE-DERIVED EXTRACELLULAR VESICLES

(71) Applicant: The Seventh Affiliated Hospital of Sun Yat sen University (Shenzhen), Shenzhen (CN)

(72) Inventors: Mingyu Huo, Shenzhen (CN); Huaqi Zhang, Foshan (CN); Changhua Zhang, Shenzhen (CN)

(21) Appl. No.: 18/540,868

(22) Filed: Dec. 15, 2023

Related U.S. Application Data
(63) Continuation of application No. PCT/CN2022/138912, filed on Dec. 14, 2022.

(30) Foreign Application Priority Data
Jun. 22, 2022 (CN) 2022107160561

(51) Int. Cl. C12N 5/09; C12M 1/00

(52) U.S. Cl. CPC C12N 5/0693; C12M 47/04; C12N 2509/00

(57) ABSTRACT

A method for separating and extracting subpopulations of gastric cancer tissue-derived extracellular vesicles includes the following steps: to first immerse gastric cancer tumor tissues into a digestive solution for enzymatic digestion to obtain a tissue digestion supernatant, simultaneously, to formulate different digestive solutions to optimize the enzymatic digestion process, followed by performing multiple differential centrifugations on the tissue digestion supernatant to obtain centrifuged supernatants respectively, thereby obtaining the tissue-derived extracellular vesicles and their subpopulations. This separation and extraction method is low-cost, reagents are easy to obtain, simple to formulate, and the brand can be selected from a wide range, and the digestive solution with collagenase II and deoxyribonuclease I (DNaseI) has a higher extraction efficiency than that with other digests, which can more realistically reflect the biological functions and modes of action of the tissue-derived extracellular vesicles.

### TECHNICAL FIELD

The disclosure relates to the technical field of biomedicine, and particularly to a method for separating and extracting subpopulations of gastric cancer tissue-derived extracellular vesicles.

### BACKGROUND

Extracellular vesicles (EVs) are mostly 30 nanometer (nm)-150 nm in diameters and have lipid bilayers. EVs are secreted by various cells in vivo and contain various bioactive substances such as proteins and ribonucleic acid (RNA) derived from mother cells. EVs, which can participate in processes such as cell communication, cell migration, angiogenesis, and tumor cell growth, are widely present in various bodily fluids and interstitial spaces, and stably carry some important signaling molecules. After entering the extracellular matrix (ECM), EVs can directionally move and specifically bind to target cells, activate intracellular signaling cascade by binding to surface receptors of the target cells. EVs can also release internal bioactive substances directly into the target cells through vesicle internalization and fusion, leading to changes in biological functions of the target cells and achieving corresponding regulatory effects. The biological effects of EVs are extensive, and the specific bioactive substances carried by EVs from different cells can play important regulatory roles in different environments. In addition, due to the stable lipid bilayers of EVs, EVs can resist most degradation and destruction, and have good transport potential. In current research, most EVs are obtained through the secretion of cell lines, and exploring the pathophysiological functions of EVs on the organism has important guiding values in fields such as tumor vaccines, biomarkers, chemotherapeutics carriers, and targeted biological therapies. However, the composition of cells in human tissues is very complex, and multiple cells can form the human tissue through a certain distribution to perform normal physiological functions. Therefore, EVs extracted from cell lines cultured in vitro cannot truly reflect the biological functions of EVs in vivo.

In recent years, due to the fact that tissue-derived extracellular vesicles can be directly separated and extracted from tissue samples, and through subpopulation classification, proteomics, and transcriptomics testing of the extracted EVs, more pathophysiological information contained in the tissue-derived extracellular vesicles can be discovered, which can realistically reflect the biological functions and modes of action of tumor-derived extracellular vesicles, these findings have gradually promoted the concept of the tissue-derived extracellular vesicles. However, as an emerging field of EVs research, the extraction technology of the tissue-derived extracellular vesicles is still in the exploratory stage, there is no accurate and efficient extraction technology for various malignant tumor tissue-derived extracellular vesicles, and the specific composition of the tissue-derived extracellular vesicles is also not yet clear. At present, there are two main methods for extracting the tissue-derived extracellular vesicles: enzyme digestion and lixiviation process. However, due to the high cost of using size exclusion chromatography (SEC), ultrafiltration tubes, and digestive enzymes, complex reagent applications, and cumbersome operational steps, the two main methods cannot be widely applied. In the existing studies, it has been found that the tissue-derived extracellular vesicles of renal carcinoma and melanoma are not a unified whole, and there are multiple subpopulations and clusters. As one of the malignant tumors with high incidence rate, the gastric cancer has not yet defined the subpopulations and clusters of the gastric cancer tissue-derived extracellular vesicles, and there is no relevant extraction technical standard. Therefore, if multiple subpopulations of the gastric cancer tissue-derived extracellular vesicles can be separated and extracted, it can truly reflect the biological functions and modes of action of the gastric cancer tissue-derived extracellular vesicles.

### SUMMARY

The disclosure optimizes an enzymatic digestion process by preparing a digestive solution, and combines differential centrifugation to separate and extract multiple subpopulations of gastric cancer tissue-derived extracellular vesicles, in order to solve the problem of fewer separation and extraction methods and high cost for the subpopulations of the gastric cancer tissue-derived extracellular vesicles.

In order to achieve the above objectives, the technical solutions of the disclosure are as follows.

A method for separating and extracting subpopulations of gastric cancer tissue-derived extracellular vesicles includes steps 1 to 4:
step 1: gastric cancer tissues are immersed into a digestive solution for an enzymatic digestion to obtain digested gastric cancer tissues, and the digested gastric cancer tissues are filtrated to obtain a tissue digestion supernatant. The digestive solution is a mixture of collagenase II and deoxyribonuclease I, a mixture of collagenase IV and deoxyribonuclease I, or a combined solution of a mixture of ethylenediaminetetraacetic acid (EDTA) and dithiothreitol with collagenase IV;
step 2: the tissue digestion supernatant from step 1 is taken to centrifuge at 500-1000× gravitational force (g) for 10-15 minutes at 4° C. to obtain a first centrifuged supernatant, and the first centrifuged supernatant is taken to centrifuge at 3000-5000×g for 15-20 minutes at 4° C. to obtain a second centrifuged supernatant;
step 3: the second centrifuged supernatant from step 2 is taken to ultra-centrifuge at 15000-17000×g for 15-30 minutes at 4° C., followed by collecting and filtering to obtain a third centrifuged supernatant, and the third centrifuged supernatant is taken to ultra-centrifuge at 50000-120000×g for 60-100 minutes at 4° C., followed by filtering to obtain a first precipitate and a fourth centrifuged supernatant, and the first precipitate is first subpopulation of the gastric cancer tissue-derived extracellular vesicles; and
step 4: the fourth centrifuged supernatant from step 3 is taken to ultra-centrifuge at 150000-200000×g for 60-100 minutes at 4° C., followed by filtering to obtain a second precipitate, and the second precipitate is second subpopulation of the gastric cancer tissue-derived extracellular vesicles.

In an embodiment, each of the first and second subpopulations of the gastric cancer tissue-derived extracellular vesicles can be applied to treat gastric cancer, for example, it can be as a target, a drug delivery tool, and/or a biomarker, it can also be prepared to a therapeutic agent.

In an embodiment, the digestive solution is the mixture of collagenase II and deoxyribonuclease I.

In an embodiment, step 2 includes: the tissue digestion supernatant from step 1 is taken to centrifuge at 500×g for 10 minutes at 4° C. to obtain the first centrifuged supernatant, and the first centrifuged supernatant is taken to centrifuge at 3000×g for 20 minutes at 4° C. to obtain the second centrifuged supernatant.

In an embodiment, the step 3 includes: the second centrifuged supernatant is taken to ultra-centrifuge at 16500×g for 30 minutes at 4° C., followed by collecting and filtering to obtain the third centrifuged supernatant, and the third centrifuged supernatant is taken to ultra-centrifuge at 100000×g for 60 minutes at 4° C., followed by filtering to obtain the first precipitate.

In an embodiment, the step 4 includes: the fourth centrifuged supernatant is taken to ultra-centrifuge at 160000×g for 90 minutes at 4° C., followed by filtering to obtain the second precipitate.

In an embodiment, a volume ratio of the collagenase II and the deoxyribonuclease I is 1:1 when the tissue digestive solution is the mixture of collagenase II and deoxyribonuclease I, or a volume ratio of the collagenase IV and the deoxyribonuclease I is 1:1 when the tissue digestive solution is the mixture of collagenase IV and deoxyribonuclease I.

In an embodiment, the enzymatic digestion in step 1 includes: digestion for 20-80 minutes in an incubation shaker at 37° C. and 100-400 revolutions per minutes (rpm).

In an embodiment, a concentration of the collagenase II in the digestive solution is 2 (milligrams per milliliter) mg/mL, and a concentration of the deoxyribonuclease I in the digestive solution is 0.2 mg/mL when the tissue digestive solution is the mixture of collagenase II and deoxyribonuclease I.

In an embodiment, a concentration of the collagenase IV in the digestive solution is 2 mg/mL, and a concentration of the deoxyribonuclease I in the digestive solution is 0.2 mg/mL when the tissue digestive solution is the mixture of collagenase IV and deoxyribonuclease I.

In an embodiment, the first subpopulation and the second subpopulation are two completely independent subpopulations.

The beneficial effects of the disclosure are as follow.

The disclosure separates and extracts various subpopulations of gastric cancer tissue-derived extracellular vesicles by immersing gastric cancer tissues in the digestive solution for the enzymatic digestion, and then extracting them by differential centrifugation for many times. This separation and extraction method uses two kinds of enzymes with low cost, easy-to-obtain reagents, simple preparation, and a wide range of brand options, and greatly reduces the operation difficulty and cost on the basis of ensuring the quantity and quality of the extracellular vesicles. Simultaneously, the prepared digestive solution has higher extraction efficiency than other digestive solutions. Compared with the related art in which a digestive enzyme formula is used for tissues of multiple sources, the disclosure can realistically reflect the biological functions and modes of action of the gastric cancer tissue-derived extracellular vesicles.

### BRIEF DESCRIPTION OF DRAWINGS

In order to provide a clearer explanation of the technical solutions of the disclosure, a brief introduction will be given to the attached drawings required for embodiments. It is evident that the attached drawings in the following description are only some of the embodiments of the disclosure. For those skilled in the art, other accompanying drawings can be obtained based on these drawings without any creative effort.

FIG. 1A illustrates a schematic diagram of particle concentrations of high-density extracellular vesicles (EV-HD) subpopulations extracted by a digestive solution of collagenase II+deoxyribonuclease I (DNase I) measured by nanoparticle tracking analysis (NTA) according to an embodiment 1 of the disclosure.

FIG. 1B illustrates a schematic diagram of particle concentrations of low-density extracellular vesicles (EV-LD) subpopulations extracted by the digestive solution of collagenase II+DNase I measured by NTA according to the embodiment 1 of the disclosure.

FIG. 1C illustrates a schematic diagram of particle concentrations of EV-HD subpopulations extracted by a digestive solution of collagenase IV+DNase I measured by NTA according to an embodiment 2 of the disclosure.

FIG. 1D illustrates a schematic diagram of particle concentrations of EV-LD subpopulations extracted by the digestive solution of collagenase IV+DNase I measured by NTA according to the embodiment 2 of the disclosure.

FIG. 2A illustrates a schematic diagram of particle concentrations of EV-HD subpopulations extracted by a digestive solution of collagenase XI+Dispase II measured by NTA according to an embodiment 3 of the disclosure.

FIG. 2B illustrates a schematic diagram of particle concentrations of EV-HD subpopulations extracted by a digestive solution of ethylenediaminetetraacetic acid/dithiothreitol (EDTA/DTT)+collagenase IV measured by NTA according to an embodiment 4 of the disclosure.

FIG. 2C illustrates a schematic diagram of particle concentrations of EV-LD subpopulations extracted by the digestive solution of collagenase XI+Dispase II measured by NTA according to the embodiment 3 of the disclosure.

FIG. 2D illustrates a schematic diagram of particle concentrations of EV-LD subpopulations extracted by the digestive solution of EDTA/DTT+collagenase IV measured by NTA according to the embodiment 4 of the disclosure.

FIG. 3 illustrate a structural diagram of the EV-HD subpopulation and EV-LD subpopulation extracted from the different digestive solutions in the embodiments 1 to 4 at 200 nanometer (nm) and 500 nm, A1 and A2 represent the digestive solution of collagenase II+DNase I in the embodiment 1, B1 and B2 represent the digestive solution of collagenase IV+DNase I in the embodiment 2, C1 and C2 represent the digestive solution of collagenase XI+Dispase II in the embodiment 3, D1 and D2 represent the digestive solution of EDTA/DTT+collagenase IV in the embodiment 4, A1/B1/C1/D1 represent the EV-HD subpopulations, and A2/B2/C2/D2 represent the EV-LD subpopulations.

FIG. 4A illustrates a histogram of protein contents of EV-HD subpopulation extracted from the different digestive solutions in the embodiments 1 to 4, scheme A represents the digestive solution of collagenase II+DNase I in the embodiment 1, scheme B represents the digestive solution of collagenase IV+DNase I in the embodiment 2, scheme C represents the digestive solution of collagenase XI+Dispase II in the embodiment 3, and scheme D represents the digestive solution of EDTA/DTT+collagenase IV in the embodiment 4.

FIG. 4B illustrates a number diagram of vesicles obtained per gram of tissues of EV-HD subpopulation extracted from the different digestive solutions in the embodiments 1 to 4, scheme A represents the digestive solution of collagenase II+DNase I in the embodiment 1, scheme B represents the digestive solution of collagenase IV+DNase I in the embodiment 2, scheme C represents the digestive solution of collagenase XI+Dispase II in the embodiment 3, and scheme D represents the digestive solution of EDTA/DTT+collagenase IV in the embodiment 4.

FIG. 4C illustrates a histogram of protein contents of EV-LD subpopulation extracted from the different digestive solutions in the embodiments 1 to 4, scheme A represents the digestive solution of collagenase II+DNase I in the embodiment 1, scheme B represents the digestive solution of collagenase IV+DNase I in the embodiment 2, scheme C represents the digestive solution of collagenase XI+Dispase II in the embodiment 3, and scheme D represents the digestive solution of EDTA/DTT+collagenase IV in the embodiment 4.

FIG. 4D illustrates a number diagram of vesicles obtained per gram of tissues of EV-LD subpopulation extracted from the different digestive solutions in the embodiments 1 to 4, scheme A represents the digestive solution of collagenase II+DNase I in the embodiment 1, scheme B represents the digestive solution of collagenase IV+DNase I in the embodiment 2, scheme C represents the digestive solution of collagenase XI+Dispase II in the embodiment 3, and scheme D represents the digestive solution of EDTA/DTT+collagenase IV in the embodiment 4.

FIG. 5 illustrates an expression of specific biomarkers for the EV-HD subpopulation and EV-LD subpopulation extracted from the different digestive solutions in the embodiments 1 to 4, A1 and A2 represent the digestive solution of collagenase II+DNase I in the embodiment 1, B1 and B2 represent the digestive solution of collagenase IV+DNase I in the embodiment 2, C1 and C2 represent the digestive solution of collagenase XI+Dispase II in the embodiment 3, D1 and D2 represent the digestive solution of EDTA/DTT+collagenase IV in the embodiment 4, A1/B1/C1/D1 represent the EV-HD subpopulations, A2/B2/C2/D2 represent the EV-LD subpopulations.

FIG. 6 illustrates a protein expression clustering heat map of the EV-HD subpopulation and EV-LD subpopulation.

FIG. 7 illustrates a biological functional analysis diagram of differentially expressed proteins in extracellular vesicle subpopulations.

### DETAILED DESCRIPTION OF EMBODIMENTS

The following is clear and complete descriptions of the technical solutions in the embodiments of the disclosure. It should be noted that the explanations of these embodiments are intended to assist in understanding the disclosure, but does not constitute a limitation of the disclosure. In addition, the technical features involved in the various embodiments of the disclosure described below can be combined with each other as long as they do not conflict with each other. The experimental methods in the following embodiments are conventional unless otherwise specified.

### Embodiment 1

A method for separating and extracting subpopulations of gastric cancer tissue-derived extracellular vesicles includes the following steps.
1. Fresh tumor tissues from postoperative tumor pathological specimens of a gastric cancer patient are enzymatically digested within 2 hours after a surgery to obtain a tissue digestion supernatant.
2. Differential centrifugations are performed with the tissue digestion supernatant to obtain gastric cancer tissue-derived extracellular vesicles and subpopulations of the gastric cancer tissue-derived extracellular vesicles.

The postoperative tumor pathological specimens used in the disclosure are all fresh tumor pathological specimens from patients undergoing radical gastrectomy for gastric cancer at the Seventh Affiliated Hospital of Sun Yat-sen University (Shenzhen). Sampling is completed within 2 hours after the specimens are isolated, and the samples are briefly stored in phosphate buffered saline (PBS) for precooled at 4° C., transported in an ice box, and the samples are processed within 4 hours.

The specific steps of separation and extraction include the following steps (1) to (9).

In the step (1), 0.5 gram (g)-1 g of the fresh tumor tissues from postoperative tumor pathological specimens of the gastric cancer patient are taken to be cleaned with PBS for three times, after the cleaning, excess adipose tissues and connective tissues are removed from the gastric cancer tissues, and followed by using tissue scissors to cut the gastric cancer tissues into small pieces with a diameter of 1-2 millimeter (mm).

In the step (2), a digestive solution of collagenase II and DNase I is prepared:
\textcircled{1} 100 milligrams (mg) of collagenase II (Yeasen Biotechnology Shanghai Co., Ltd.) are added to 500 microliter (μL) of hank's balanced salt solution (HBSS) buffer to prepare 200 mg/mL of collagenase II storage solution, and the collagenase II storage solution is stored for a long time at −20° C.;
\textcircled{2} a mixed solution with a potential of hydrogen (pH) of 6.5 and containing 20 millimoles per liter (mM) of sodium acetate, 5 mM of calcium chloride (CaCl₂), 0.1 mM phenyl methane sulfonyl fluoride (PMSF), and glycerin equivalent to 50% of the mixed solution is prepared. Then, DNase I powder is taken to dissolve in 1 mL of the mixed solution to obtain a DNase I storage solution;
\textcircled{3} the collagenase II storage solution is diluted to 4 mg/mL with the HBSS buffer, the DNase I storage solution is diluted to 400 micrograms per milliliter (m/mL) with 0.15 moles per liter (M) of NaCl solution, after diluting, the diluted collagenase II storage solution and the diluted DNase I storage solution are finally mixed in a ratio of 1:1 to obtain the digestive solution with a final concentration of collagenase II of 2 mg/mL and DNase I of 0.2 mg/mL.

In the step (3), the cut small pieces of the fresh gastric cancer tissues from step (1) are immersed in 10 mL of the digestive solution prepared in step (2), followed by placing in a 37° C. constant temperature shaker to digest at 200 revolutions per minute (rpm) for 60 minutes, and then filtering with a 40 micrometers (μm) filter to obtain a tissue digestion supernatant of the fresh gastric cancer tissues.

In the step (4), the filtered tissue digestion supernatant of the fresh gastric cancer tissues in step (3) is centrifuged in a frozen centrifuge at 500×g for 10 minutes at 4° C., and followed by filtering to obtain a first centrifuged supernatant;

In the step (5), the first centrifuged supernatant from step (4) is centrifuged in the frozen centrifuge at 3000×g for 20 minutes at 4° C., and followed by filtering to obtain a second centrifuged supernatant.

In the step (6), the second centrifuged supernatant from step (5) is centrifuged in a ultracentrifuge at 16500×g for 30 minutes at 4° C., followed by filtering with a 0.8 μm filter to obtain a third centrifuged supernatant.

In the step (7), the third centrifuged supernatant from step (6) is centrifuged in the ultracentrifuge at 100000×g for 60 minutes at 4° C., followed by filtering to obtain a first precipitate and a fourth centrifuged supernatant, the first precipitate is the first subpopulation of the gastric cancer tissue-derived extracellular vesicles, and titled as EV-HD subpopulation.

In the step (8), the fourth centrifuged supernatant from step (7) is centrifuged again in ultracentrifuge at 160000×g for 90 minutes at 4° C., followed by filtering to obtain a second precipitate, and the second precipitate is the second subpopulation of the gastric cancer tissue-derived extracellular vesicles, and titled as EV-LD subpopulation.

In the step (9), the first precipitate and the second precipitate obtained from steps (7) and (8) are resuspended with the PBS solution to obtain different subpopulation suspensions of the gastric cancer tissue-derived extracellular vesicles for subsequent experiments.

### Embodiment 2

The method for separating and extracting subpopulations of the gastric cancer tissue-derived extracellular vesicles includes the following steps.

The specific steps of separation and extraction are the same as that of the embodiment 1, and the difference is that the digestive solution used in the embodiment 2 is a digestive solution of collagenase IV+DNase I, 100 mg of the collagenase IV storage solution is diluted to 4 mg/mL with the HBSS buffer, after the diluting, the diluted collagenase IV storage solution and the DNase I storage solution diluted to 400 μg/mL in the embodiment 1 are finally mixed in a ratio of 1:1 to obtain the digestive solution with a final concentration of collagenase IV of 2 mg/mL and DNase I of 0.2 mg/mL.

### Embodiment 3

The method for separating and extracting subpopulations of the gastric cancer tissue-derived extracellular vesicles includes the following steps.

The specific steps of separation and extraction are the same as that of the embodiment 1, and the difference is that the digestive solution used in the embodiment 3 is a digestive solution of Dispase II+collagenase XI, the collagenase XI storage solution with a concentration of 1 mg/mL is prepared through the collagenase XI and the HBSS buffer, the Dispase II storage solution with a concentration of 1 mg/mL is prepared through the Dispase II and the HBSS buffer, and the collagenase XI storage solution and the Dispase II storage solution are finally mixed in a ratio of 1:1 to obtain the digestive solution.

### Embodiment 4

The method for separating and extracting subpopulations of the gastric cancer tissue-derived extracellular vesicles includes the following steps.

The specific steps of separation and extraction are the same as that of the embodiment 1, and the difference is that the digestive solution used in the embodiment 4 is a digestive solution of ethylenediaminetetraacetic acid (EDTA)+dithiothreitol (DTT)+collagenase IV, 1-2 mm of the cut small pieces of the fresh gastric cancer tissues are immersed in 10 mL of the mixed solution (prepared by 1.0 M EDTA and 0.1 M DTT), followed by placing in the 37° C. constant temperature shaker to digest at 200 rpm for 30 minutes, and then the digested small pieces of the gastric cancer tissue are immersed in 10 mL of the diluted collagenase IV storage solution (preparation of the diluted collagenase IV storage solution is same as that of the embodiment 2), followed by placing in the 37° C. constant temperature shaker to digest at 200 rpm for 60 minutes, and then filtering with the 40 μm filter to obtain the tissue digestion supernatant of the fresh gastric cancer tissues.

### Experiment 1

The effects of different digestive solutions on efficiencies of extracting tissue-derived extracellular vesicles are analyzed.

1. Detecting Particle Sizes and Concentrations of Extracellular Vesicles

The EV-HD subpopulations and the EV-LD subpopulations extracted from the embodiments 1 to 4 are separately taken 10 μL to dilute with the PBS buffer to 30 μL, and then a particle size analyzer (NanoFCM, N30E) is used to detect the diluted extracellular vesicle subpopulations.

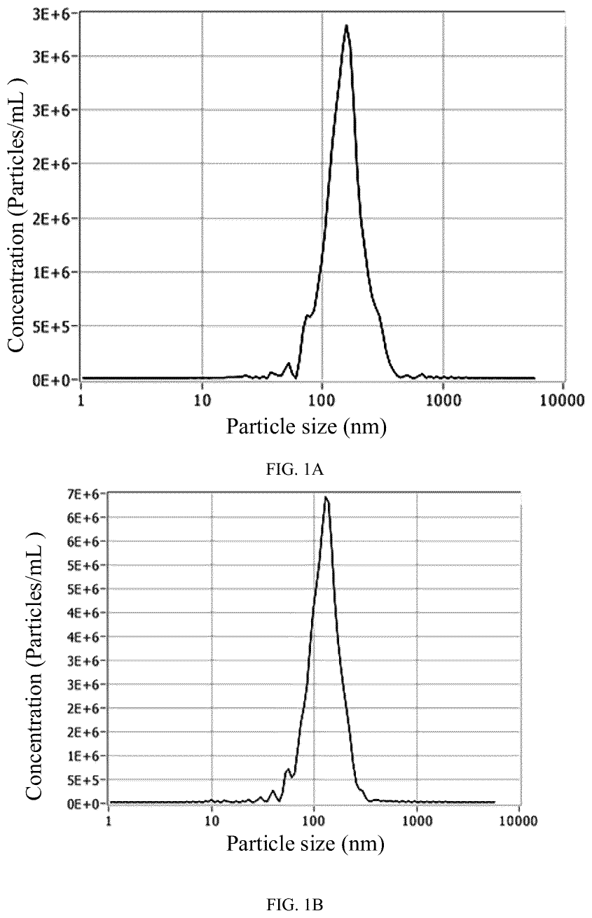

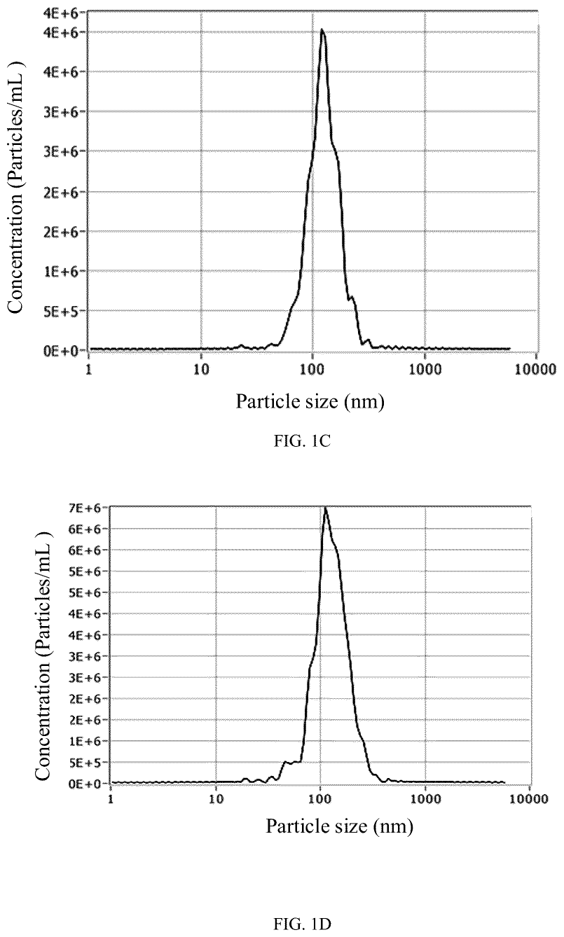

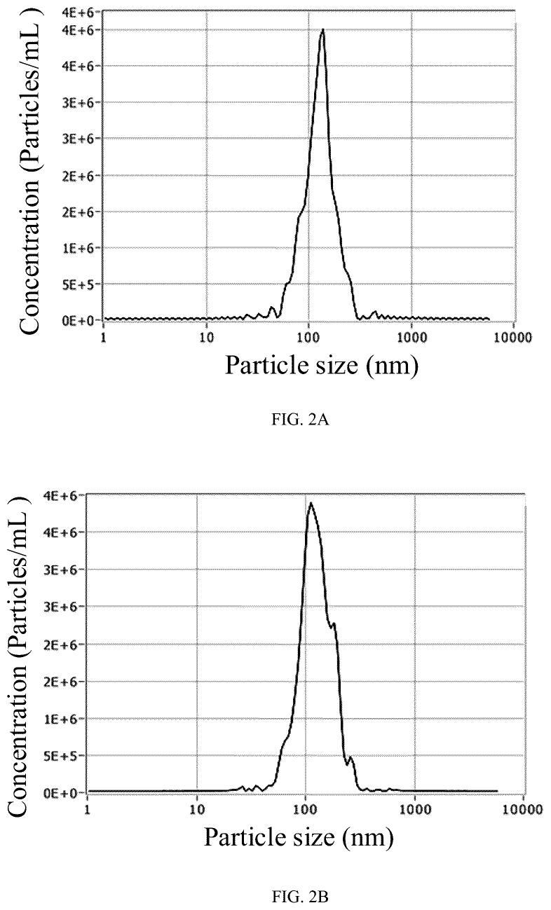

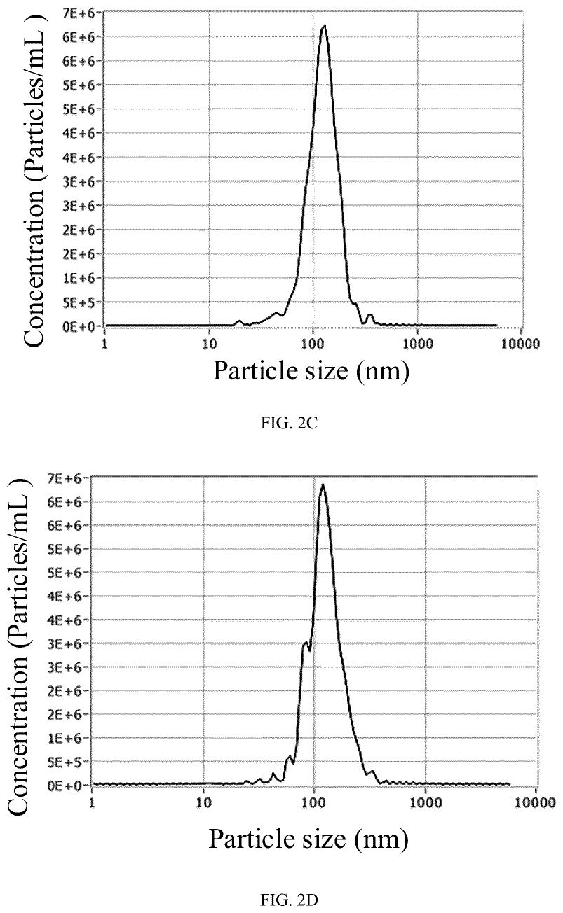

As shown in FIGS. 1A to 2D, sizes of the extracellular vesicles extracted from the embodiments 1 to 4 are mainly in a range of 50 nm to 200 nm.

2. Transmission Electron Microscopy Detection

The EV-HD subpopulations and the EV-LD subpopulations extracted from the embodiments 1 to 4 are separately taken 10 μL to drop onto a copper mesh to precipitate for 1 minute, and followed by removing floating liquids by suctions with filter papers. After that, 10 μL of uranyl acetate are used to drop on the copper mesh to precipitate for 1 minute, and followed by removing floating liquids by suctions with the filter papers. After drying at room temperature for 5-10 minutes, a transmission electron microscopy (Hitachi, H-7650) is used to perform electron microscopy imaging under a voltage of 100 kilovolts (kv) with a field of view of 200 nm and 500 nm, respectively.

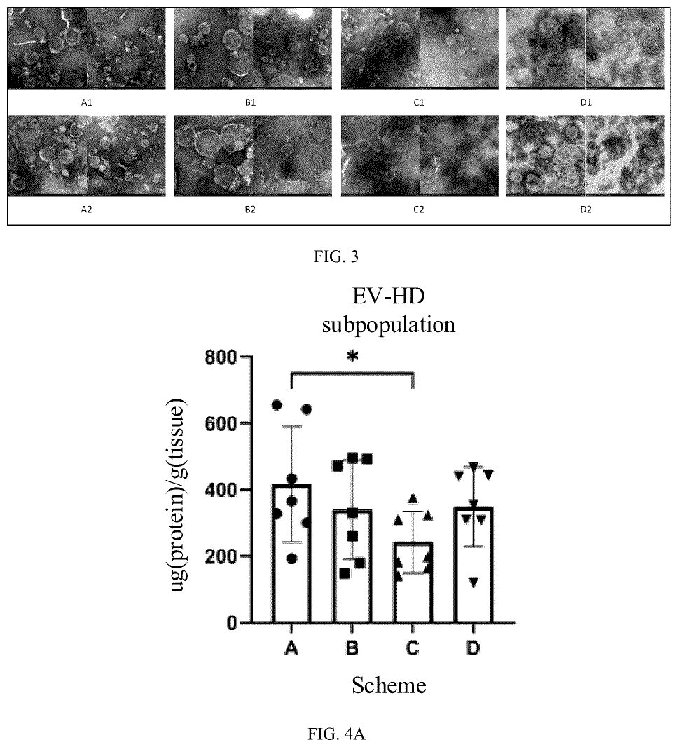

As shown in FIG. 3, A1, B1, C1, and D1 are the EV-HD subpopulations, A2, B2, C2, and D2 are the EV-LD subpopulations, which illustrates that tissue-derived extracellular vesicles obtained in the embodiments 1 to 4 have lipid bilayers.

3. Analyzing Protein Contents, Number of Vesicles, and Western Blotting Validation of Subpopulations of the Extracellular Vesicles

The specific steps for analyzing protein contents, number of vesicles, and western blotting validation of subpopulations of the extracellular vesicles are as follows.

(1) Extracting Whole Protein of Extracellular Vesicles

According to the instructions of the whole protein extraction kit (beyotime biotechnology), 1 mL of lysis buffer, 10 μL of 100× phosphatase inhibitor, 10 μL 100× protease inhibitor and 5 μL of PMSF are mixed to prepare a lysis solution. 50 μL of EV-HD subpopulations and EV-LD subpopulations extracted from the embodiments 1 to 4 are individually taken to add 10 μL of the lysis solution to obtain multiple mixtures, the multiple mixtures are lysed on ice for 30 minutes, with shaking, during the lysis process, for 30 seconds every 4 minutes and then standing on the ice, to obtain lysed mixtures. After the lysis process, the lysed mixtures are centrifuged at 12000×g for 5 minutes at 4° C. to obtain centrifuged supernatants corresponding to the lysed mixtures, and the centrifuged supernatant is the whole protein extract. Then a 5× sample buffer solution with a volume of 25% of the whole protein extract is added to each whole protein extract, followed by mixing thoroughly to denature the protein in each whole protein extract in a 99° C. metal bath for 5-10 minutes to obtain the denatured protein, and the denatured protein is stored in an environment of −80° C.

(2) Detection of Protein Content and Number of Vesicles

The 96-well microtiter trays are taken to add with 10 μL of the protein standard reagent in accordance with the instructions of the protein content assay kit (beyotime biotechnology), and the protein standard reagent in the 96-well microtiter trays is diluted with deionized water to 100 μL to make a final concentration of the protein standard reagent be 0.5 mg/mL. 2 μL of the denatured proteins from the EV-HD and EV-LD subpopulations extracted from step 1 are separately taken and diluted with the deionized water to 20 μL. Then, a bicinchoninic acid assay (BCA) working solution is prepared with a ratio of 50:1 of the reagent A in the BCA reagent and the reagent B in the BCA reagent. 200 μL of the BCA working solution are added into each well of the 96-well microtiter trays, followed by mixing thoroughly to incubate at 37° C. for 30 minutes. An absorbance value is then recorded with the aid of an enzyme marker (BioTek, Synergy H1M) and a standard curve is plotted to determine the protein content of the extracellular vesicle subpopulations.

In addition, the particle size analyzer (NanoFCM, N30E) is used to measure the concentration of the extracellular vesicles, and then the total volume is used to calculate the number of vesicles obtained in the embodiments 1 to 4, which in turn is calculated to obtain the number of vesicles obtained from per gram of tumor tissue in the embodiments 1 to 4.

Experimental Result:

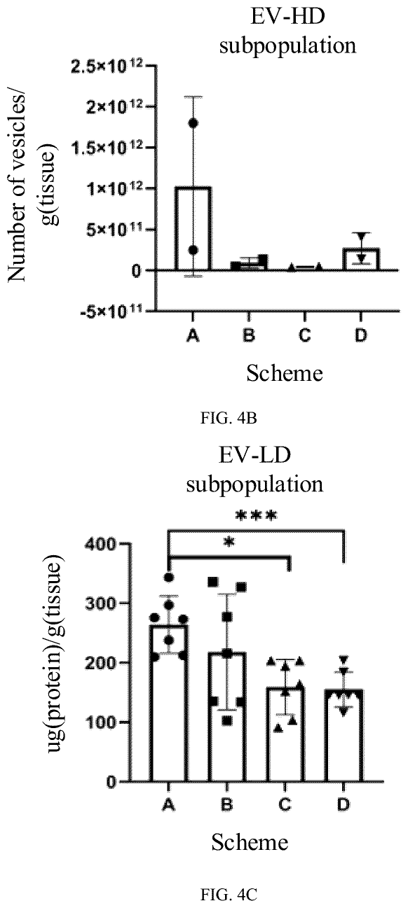

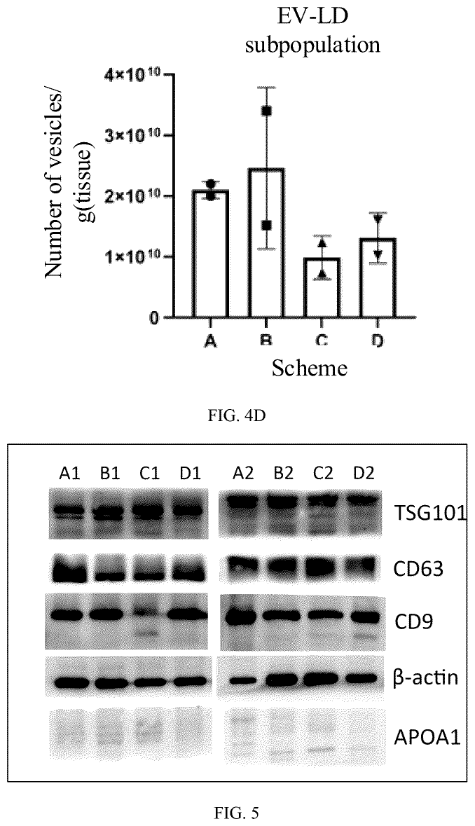

As shown in FIGS. 4A to 4D, the scheme A is the subpopulation of extracellular vesicles extracted from the digestive solution of collagenase II+DNase I in the embodiment 1, the scheme B is the subpopulation of extracellular vesicles extracted from the digestive solution of collagenase IV+DNase I in the embodiment 2, the scheme C is the subpopulation of extracellular vesicles extracted from the digestive solution of collagenase XI+Dispase II in the embodiment 3, and the scheme D is the subpopulation of extracellular vesicles extracted from the digestive solution of EDTA/DTT+collagenase IV+DNase I in the embodiment 4.

When extracting EV-HD subpopulation, the scheme A has the highest protein content and the number of vesicles obtained per gram of tissue, while the scheme C has the lowest protein content and the number of vesicles obtained per gram of tissue. When extracting the EV-LD subpopulation, the scheme A has the highest protein content and the scheme D has the lowest protein content. Although the scheme B obtains more vesicles of subpopulation EV-LD from per gram of tissue than the scheme A, the scheme A extracts more vesicles of EV-HD subpopulation and EV-LD subpopulation in total than the scheme B. In summary, the digestive solution prepared with collagenase II and DNase I to extract extracellular vesicle subpopulations in the embodiment 1 has the highest efficiency, and is the optimal extraction scheme.

(3) Western Blotting Experiment

The specific steps for the western blotting experiment are as follows.
\textcircled{1} Containers by manually encapsulating glue and glass plates are prepared. After the containers and the glass plates are fixed and are confirmed with no water leakage, gels are prepared according to the following ingredients:
5% concentrated gel: 2.7 mL of H₂O, 0.67 mL of 30% acrylamide, 0.5 mL of 1.5 M Tris-HCl (pH=6.8), 0.4 mL of 10% sodium dodecyl sulfate (SDS), 0.4 mL of 10% ammonium persulfate substitute (APS), and 0.004 mL of tetramethylethylenediamine (TEMED)
8% separation gel: 4.6 mL of H₂O, 2.7 mL of 30% acrylamide, 2.5 mL of 1.5 M Tris-HCl (pH=8.8), 0.1 mL of 10% SDS, 0.1 mL of 10% APS, and 0.006 mL of TEMED
10% separation gel: 4 mL of H₂O, 3.3 mL of 30% acrylamide, 2.5 mL of 1.5 M Tris-HCl (pH=8.8), 0.1 mL of 10% SDS, 0.1 mL of 10% APS, and 0.004 mL of TEMED.
12% separation gel: 3.3 mL of H₂ O, 4 mL of 30% acrylamide, 2.5 mL of 1.5 M Tris-HCl (pH=8.8), 0.1 mL of 10% SDS, 0.1 mL of 10% APS, and 0.006 mL of TEMED.
\textcircled{2} The prepared separation gels are respectively added into the containers to a certain height to ensure that upper edges of the separation gels are about 1 cm away from the lower edge of the combs. Then 1 mL of absolute ethanol is added to upper layers of the separation gels, followed by waiting for 30 minutes until the separation gels are completely solidified and then removing the absolute ethanol. Then 5% concentrated gel is added into the upper layer of the separation gels, and the combs are inserted, followed by waiting for 25 minutes until the concentrated gels are completely solidified to obtain first gels.
\textcircled{3} The first gels prepared from step \textcircled{2} are added into an electrophoresis tank, then sufficient electrophoresis buffer is added and the comb is pulled out from the electrophoresis buffer, followed by adding immunoblotting indicator and adding 15 μg of denatured proteins from the EV-HD and EV-LD subpopulations extracted in step (1) into each hole.
\textcircled{4} A fixed voltage of 80 voltages (V) is set for concentrating the gels, and the voltage is switched after the protein strip runs through the concentrated gels. The gel electrophoresis is carried out at the fixed voltage of 120V for about 70 minutes to concentrate the gels until the immunoblotting indicator runs to the target position, and then the gel electrophoresis is stopped.
\textcircled{5} The sponges and filter papers are placed on both sides of transmembrane clamps, and polyvinylidene difluoride (PVDF) membranes are cut to an appropriate size of 0.45 μm and put in methanol to activate for 1 minute, followed by stabilizing in transmembrane solutions for 3 minutes. The electrophoresis gel after the electrophoresis and the PVDF membrane are placed in directions of electrodes, the air bubbles are discharged, the transmembrane clamps are assembled and fixed in transmembrane tanks, then a pre-cooled transmembrane buffer is added, the transmembrane tanks are placed in the ice and set a fixed current of 200 milliampere (mA), a transmembrane time is 70 minutes, the PVDF membrane is taken out, and the corresponding positions of the PVDF membrane are cut according to the molecular weight of proteins and washed with tris buffered saline with Tween® 20 (TBST) buffer for 3 times, each time for 3 minutes.
\textcircled{6} The containment solution containing 5% skimmed milk powder is prepared with TBST buffer, and the PVDF membrane is immerged into the containment solution and closed at room temperature for 1 hour. After the closing, the PVDF membrane is washed with TB ST buffer for 3 times, each time for 10 min.
\textcircled{7} The preparation of primary antibody working solution: the primary antibodies are the corresponding antibodies of target proteins TSG101, CD63, CD9, β-actin, APOA1, including Rabbit-TSG101 Antibody (Abcam©, USA), Rabbit-CD63 Antibody (Abcam©, USA), Rabbit-CD9 Antibody (Abcam©, USA), Rabbit-β-actin Antibody (Proteintech©, USA), Rabbit-APOA1 Antibody (Affinity©, USA), respectively. The primary antibody stock solution and primary antibody diluent (Solarbio©, China) are taken at a volume ratio of 1:1000 to prepare a primary antibody working solution that bind specifically to the target protein, then the washed strips are immersed in the primary antibody working solution, and incubated in a shaker at 4° C. for 12-16 hours, and the strips are taken out of the incubator after incubation, and washed with TBST buffer for 3 times, each time for 10 minutes.
\textcircled{8} The preparation of secondary antibody working solution: the secondary antibodies including horseradish peroxidase-labelled goat anti-rabbit IgG (H+L) (Beyotime©, China) and horseradish peroxidase-labelled goat anti-mouse IgG (H+L) (Beyotime©, China), respectively. The working solution of the secondary antibody that can bind specifically to the primary antibody is prepared by taking the primary solution of the secondary antibody and the dilution solution of the secondary antibody (Solarbio©, China) at a volume ratio of 1:1000, and then the cleaned strip is immersed into the working solution of the secondary antibody that corresponded to the species of the primary antibody and have a color-expression label, and incubated for 1 hour at room temperature, and then the strip is taken out of the secondary antibody after the incubation, and washed 3 times with TBST buffer, each time for 10 minutes each time.
\textcircled{9} The ultrasensitive enhanced chemiluminescence (ECL) solutions A and B are mixed at a ratio of 1:1 to formulate a luminescent working solution, and the immunoblotting bands are visualized using the ChemiDoc Touch imaging system, and the images is processed and analyzed.

Experimental result: A1, B1, C1, and D1 are EV-HD subpopulations, and A2, B2, C2, and D2 are EV-LD subpopulations, as shown in FIG. 5, the EV-HD and EV-LD subpopulations extracted from the embodiments 1-4 are seen to be expressed with specific markers TSG101, CD63, CD9, and β-actin in the extracellular vesicles, whereas the expression of negative marker APOA1 is not evident.

4. Analysis of Protein Expression Differences and Functions Between Extracellular Vesicle Subpopulations

Relative quantitative proteomics analysis is performed using the Lable-Free method, including the following steps (1) to (3).

(1) Protein Extraction and Peptide Digestion
\textcircled{1} The extracellular vesicle samples are lysed by SDT lysate (the SDT lysate is prepared from 4% (w/v) SDS, 100 mM Tris/HCl (pH=7.6), 0.1 M DTT) to extract the proteins.
\textcircled{2} Bio-Rad Protein Assay Kit is used, and the protein standard and the protein sample extracted in step 0 are added to the 96-well microtiter trays according to the instructions, the protein content of the sample is calculated after plotting the standard curve, when the protein content of the sample reached 0.05 μg/μL, the subsequent experiments can be carried out.
\textcircled{3} The four samples are set up for each of the EV-HD subpopulations and EV-LD subpopulations, and 200 μg of protein is taken from each sample and added to 30 μL of SDT buffer (made from 4% SDS, 100 mM DTT, and 150 mM Tris-HCl, pH=8.0).
\textcircled{4} The eight buffers containing the sample proteins prepared in step \textcircled{3} are added 750 μL of uric acid (UA) buffer (made from 8 M urea, 150 mM Tris-HCl, pH=8.0), and followed by repeating the ultrafiltration concentration by passing the filters (Microcon units, 10 kD) to wash out DTT and other low molecular weight components in the protein solution. After the washing, the filters are washed three times with 100 μL of UA buffer, and then two times with 100 μL of 25 mM NH₄HCO₃ solution.
\textcircled{5} After the ultrafiltration concentration in step \textcircled{4}, 100 μL of UA buffer concentrated to 100 mM by iodoacetamide are added to the protein solution concentrated by the ultrafiltration concentration in step 0, and followed by incubating for 30 min away from light to sequester residual cysteine.
\textcircled{6} The 4 μg of trypsin (Promega) are added to 40 μL of 25 mM NH₄HCO₃ solution, and then the protein solution of the sequestered cysteine closure in step \textcircled{5} is added, followed by incubating at 37° C. overnight, and finally filtering the collected filtrate after filtration with a filter to obtain a peptide.
\textcircled{7} The peptide is desalted by using C18 Cartridges, and a lyophilized peptide is obtained after a vacuum centrifugation. The lyophilized peptide is re-solubilized by using 40 μL of 0.1% formic acid solution.
\textcircled{8} The peptide is estimated by ultraviolet spectral density at 280 nm based on the frequency of tryptophan and tyrosine in vertebrate proteins calculated with an extinction coefficient of 1.1 for a 0.1% (g/L) solution.

(2) Liquid Chromatography-Mass Spectrum/Mass Spectrometry (LC-MS/MS) Data Acquisition
\textcircled{1} Each sample is separated by using high performance liquid chromatography (HPLC) liquid phase system Easy nLC with nanoliter flow rate.
\textcircled{2} A buffer A is 0.1% formic acid aqueous solution and a buffer B is 0.1% formic acid acetonitrile aqueous solution (acetonitrile is 84%).
\textcircled{3} The column is equilibrated with 95% of the buffer A. The samples are uploaded from the autosampler to the upload column (Thermo Scientific Acclaim PepMap100, 100 μm×2 cm, nanoViper C18) and separated on an analytical column (Thermo scientific EASY column, 10 cm, ID75 μm, 3 μm, C18-A2) at a flow rate of 300 NL/min.
\textcircled{4} The samples are separated by chromatography and analyzed by mass spectrometry using a Q-Exactive mass spectrometer.
\textcircled{5} A mass spectrometry detection mode is positive ion with a parent ion scanning range of 300-1800 mass-to-charge ratio (m/z), primary mass spectrometry resolution of 70,000 at 200 m/z, automatic gain control (AGC) target of 1e⁶, Maximum IT of 50 ms, and dynamic exclusion time is 60.0 s.
\textcircled{6} The mass spectrometry data is acquired by using a data-dependent top 10 method to dynamically select the most abundant precursor ions for high energy collision dissociation (HCD) fragmentation from the survey scans (300-1800 m/z).
\textcircled{7} The mass-to-charge ratios of peptides and peptide fragments are collected according to the following method: 20 fragmentation profiles are collected after each full scan (MS2 scan) with MS2 activation type is high energy collision dissociation (HCD), isolation window is 2 m/z, secondary mass spectral resolution is 17500 at 200 m/z, Normalized Collision Energy is 30 eV and Underfill is 0.1%.

(3) Protein Identification, Quantitative Analysis and Bioinformatic Analysis

The original data for mass spectrometry analysis is RAW files, and MaxQuant software (version 1.5.3.17) is used for database identification and quantitative analysis.
\textcircled{1} The protein expression difference analysis: mass spectrometry quantitative information of the samples of proteins in step (2) is normalized to an interval of (−1,1), and then the protein expression is analyzed by t-test to find out the proteins with significant expression differences.
\textcircled{2} The functional analysis: GO annotation of the target protein collection by using Blast2GO database is performed sequentially by sequence comparison (Blast), GO entry extraction (Mapping), GO annotation (Annotation), and Annotation Augmentations of InterProScan.

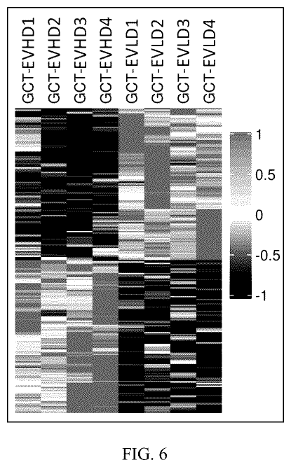

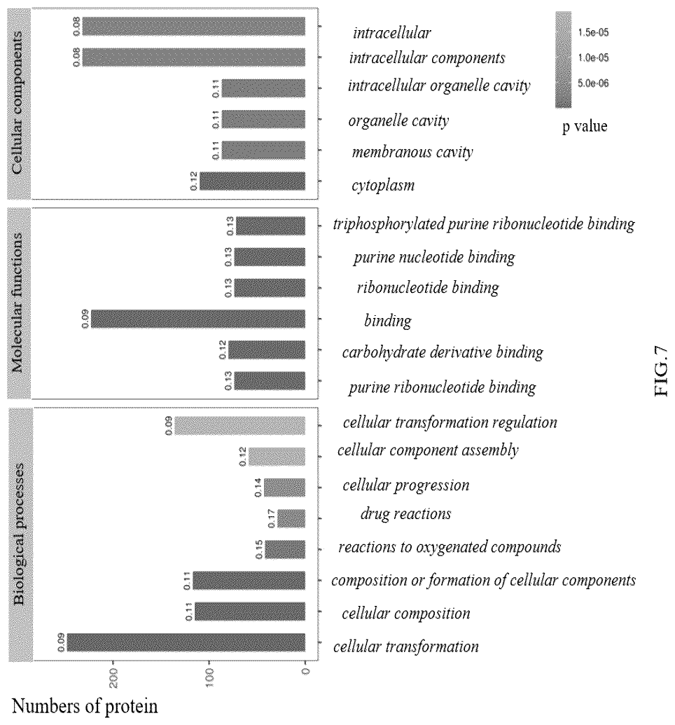

FIG. 6 illustrates the identification of protein expression differences between the two subpopulations, it can be seen that the EV-HD and EV-LD subpopulations have a variety of differentially expressed proteins, and the protein expression patterns are different. FIG. 7 illustrates the identification of the biological functions of the differentially expressed proteins between the two subpopulations, and it can be seen that the differentially expressed proteins between the two subpopulations have a variety of biological functions. Therefore, from FIG. 6 and FIG. 7, it can be seen that the EV-HD and EV-LD subpopulations are two completely independent subpopulations with different protein expression contents and different biological functions.

The above are the preferred embodiments of the disclosure. It should be pointed out that for those skilled in the art, several improvements and embellishments can be made without departing from the principles of the disclosure. These improvements and embellishments are also considered as the scope of protection of the disclosure.

What is claimed is:

1. A method for separating and extracting subpopulations of gastric cancer tissue-derived extracellular vesicles, comprising:
step 1: immersing gastric cancer tissues into a digestive solution for an enzymatic digestion to obtain digested gastric cancer tissues, and filtrating the digested gastric cancer tissues to obtain a tissue digestion supernatant; wherein the tissue digestive solution is a mixture of collagenase II and deoxyribonuclease I, a mixture of collagenase IV and deoxyribonuclease I, or a combined solution of a mixture of ethylenediaminetetraacetic acid (EDTA) and dithiothreitol with collagenase IV;
step 2: taking the tissue digestion supernatant from step 1 to centrifuge at 500-1000× gravitational force (g) for 10-15 minutes at 4° C. to obtain a first centrifuged supernatant, and taking the first centrifuged supernatant to centrifuge at 3000-5000×g for 15-20 minutes at 4° C. to obtain a second centrifuged supernatant;
step 3: taking the second centrifuged supernatant from step 2 to ultra-centrifuge at 15000-17000×g for 15-30 minutes at 4° C., followed by collecting and filtering to obtain a third centrifuged supernatant; and taking the third centrifuged supernatant to ultra-centrifuge at 50000-120000×g for 60-100 minutes at 4° C., followed by filtering to obtain a first precipitate and a fourth centrifuged supernatant; wherein the first precipitate is a first subpopulation of the gastric cancer tissue-derived extracellular vesicles; and
step 4: taking the fourth centrifuged supernatant from step 3 to ultra-centrifuge at 150000-200000×g for 60-100 minutes at 4° C., followed by filtering to obtain a second precipitate; wherein the second precipitate is a second subpopulation of the gastric cancer tissue-derived extracellular vesicles.

2. The method for separating and extracting subpopulations of gastric cancer tissue-derived extracellular vesicles as claimed in claim 1, wherein the digestive solution is the mixture of collagenase II and deoxyribonuclease I.

3. The method for separating and extracting subpopulations of gastric cancer tissue-derived extracellular vesicles as claimed in claim 1, wherein step 2 comprises:
taking the tissue digestion supernatant from step 1 to centrifuge at 500×g for 10 minutes at 4° C. to obtain the first centrifuged supernatant, and taking the first centrifuged supernatant to centrifuge at 3000×g for 20 minutes at 4° C. to obtain the second centrifuged supernatant.

4. The method for separating and extracting subpopulations of gastric cancer tissue-derived extracellular vesicles as claimed in claim 1, wherein step 3 comprises:
taking the second centrifuged supernatant to ultra-centrifuge at 16500×g for 30 minutes at 4° C., followed by collecting and filtering to obtain the third centrifuged supernatant; and taking the third centrifuged supernatant to ultra-centrifuge at 100000×g for 60 minutes at 4° C., followed by filtering to obtain the first precipitate.

5. The method for separating and extracting subpopulations of gastric cancer tissue-derived extracellular vesicles as claimed in claim 1, wherein step 4 comprises:
taking the fourth centrifuged supernatant to ultra-centrifuge at 160000×g for 90 minutes at 4° C., followed by filtering to obtain the second precipitate.

6. The method for separating and extracting subpopulations of gastric cancer tissue-derived extracellular vesicles as claimed in claim 1, wherein a volume ratio of the collagenase II and the deoxyribonuclease I is 1:1 when the tissue digestive solution is the mixture of collagenase II and deoxyribonuclease I, or a volume ratio of the collagenase IV and the deoxyribonuclease I is 1:1 when the tissue digestive solution is the mixture of collagenase IV and deoxyribonuclease I.

7. The method for separating and extracting subpopulations of gastric cancer tissue-derived extracellular vesicles as claimed in claim 1, wherein the enzymatic digestion in step 1 comprises: digestion for 20-80 minutes in an incubation shaker at 37° C. and 100-400 revolutions per minutes (rpm).

8. The method for separating and extracting subpopulations of gastric cancer tissue-derived extracellular vesicles as claimed in claim 1, wherein a concentration of the collagenase II in the digestive solution is 2 (milligrams per milliliter) mg/mL, and a concentration of the deoxyribonuclease I in the digestive solution is 0.2 mg/mL when the tissue digestive solution is the mixture of collagenase II and deoxyribonuclease I.

9. The method for separating and extracting subpopulations of gastric cancer tissue-derived extracellular vesicles as claimed in claim 1, wherein a concentration of the collagenase IV in the digestive solution is 2 mg/mL, and a concentration of the deoxyribonuclease I in the digestive solution is 0.2 mg/mL when the tissue digestive solution is the mixture of collagenase IV and deoxyribonuclease I.

10. The method for separating and extracting subpopulations of gastric cancer tissue-derived extracellular vesicles as claimed in claim 1, wherein the first subpopulation and the second subpopulation are two independent subpopulations.
